(19) United States
(12) Patent Application Publication
(10) Pub. No.: US 2001/0034320 A1
(43) Pub. Date: Oct. 25, 2001

(54) NK1-receptor antagonists for treating restless legs syndrome

(76) Inventors: Hans-Michael Brecht, Ingelheim am Rhein (DE); Birgit Jung, Schwabenheim (DE)

Correspondence Address: BOEHRINGER INGELHEIM CORPORATION, 900 RIDGEBURY ROAD, P O BOX 368, RIDGEFIELD, CT, 06877, US

(21) Appl. No.: 09/764,629

(22) Filed: Jan. 18, 2001

Related U.S. Application Data
(60) Provisional application No. 60/180,399, filed on Feb. 4, 2000.

(30) Foreign Application Priority Data
Jan. 18, 2001 (DE) DE 100 01 785 1

(51) Int. Cl. A61K 38/33; A61K 31/485; A61K 31/137

(52) U.S. Cl. 514/2; 514/282; 514/649

(57) ABSTRACT

The invention relates to the use of NK₁-receptor antagonists for preparing a pharmaceutical composition for treating Restless Legs Syndrome (RLS) and methods for treating RLS using such compounds.

### RELATED APPLICATIONS

This application claims benefit to U.S. Provisional Application No. 60/180,399 filed Feb. 4, 2000.

### TECHNICAL FIELD OF THE INVENTION

The invention relates to the use of NK₁-receptor antagonists for preparing a pharmaceutical composition for treating Restless Legs Syndrome (RLS).

### BACKGROUND OF THE INVENTION

Restless Legs Syndrome is a neurological disorder which manifests itself chiefly as sensory disorders of the legs such as tingling, dragging, tearing, itching, burning, cramp or pain and in those affected triggers an irresistible compulsion to move. Frequently these disorders occur when the affected person is resting. Particularly at night, during sleep, these sensory disorders and the consequent compulsive movements lead to restlessness and sleep disorders.

RLS occurs at all ages, increasing in frequency at more advanced ages. The prevalence in the general population is about 5%. Because of the characteristics of the symptoms RLS is one of the most common causes of sleep problems. RLS is the cause of sleeping and waking problems in 7% of 20-40 year-olds, 18% of 40-60 year-olds and 33% of over 60 year-olds.

When the patient's quality of sleep or life is increasingly affected by RLS or the patients suffer from daytime tiredness, treatment is indicated. The need for treatment generally sets in at the age of 40-50.

Hitherto there has been no permitted drug treatment available. In therapy trials, monotherapies with dopamine agonists, opiates, benzodiazepines, carbamazepine, clonidine or the combined administration of levodopa (L-dopa) in conjunction with a dopadecarboxylase inhibitor have had mixed degrees of success. Most studies have been done on the use of L-dopa in RLS. In long-term therapy, there is a significant alleviation of the complaint, with an improvement in the quality of life and sleep. The disadvantage of the L-dopa therapy, however, is that in many patients the effectiveness declines and/or there is an increase in RLS problems during the day (rebound phenomenon or augmentation).

For individual dopamine antagonists short-term therapy trials have been conducted. The dopamine agonists investigated include: bromocryptine, cabergoline, alpha-dihydroergocryptine, lisuride, pergolide, pramipexole and ropinirol.

All these dopamine antagonists were found to be effective. The results of trials on long-term therapy with dopamine antagonists are not yet available, so the question of the loss of activity after long-term use (tachyphylaxia) cannot be answered yet.

The disadvantage of the dopamine antagonists is the incidence of side-effects such as nausea, vomiting, dizziness, hypotension, constipation and sleeplessness, which generally occur initially and in dose-dependent manner.

The use of the anti-Parkinson's drug pramipexole, (S)-4,5,6,7-tetrahydro-N6-propyl-2,6-benzothiazolediamine, a D2/D3 agonist (dopamine antagonist), for treating RLS is described in WO 98/31362. Benzodiazepines and opiates are also effective in RLS. Because of the risk of dependency and the build-up of tolerance, however, these substances are only available for therapy on a restricted basis. Carbamazepine has only been tested on RLS in a few partly open trials. It gives only partial relief from the complaint and is not currently viewed as a suitable drug for treating RLS. The effect of clonidine, 2-(2,6-dichloroanilino)-4,5-dihydroimidazole, which was originally developed as an antihypertensive and miotic, in the treatment of RLS has been studied in 4 open trials, 2 double-blind, placebo-controlled trials and a single case study. The daily doses were between 0.1-0.9 mg. The patients reported a (statistically significant) reduction in perceived symptoms such as paraesthesia, compulsive movement and tiredness during the day. According to the objective polysomnographic measuring parameters, the sleep latency was indeed shortened, but the quality of sleep, frequency of waking or periodic leg movements in sleep (PLMS) were not affected. Since substances are available which are more effective as monotherapies, clonidine is currently only recommended as an alternative form of therapy under certain circumstances.

Surprisingly, it has been found that the symptoms of RLS can be suppressed by the administration of NK₁-receptor antagonists.

### DESCRIPTION OF THE INVENTION

The present invention relates to the use of NK₁-receptor antagonists for the preparation of a pharmaceutical composition for treating Restless Legs Syndrome (RLS).

It is preferable according to the invention to use an NK₁-receptor antagonist selected from among BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-I, CJ-11974, TAK-637 and GR 205171, for preparing a pharmaceutical composition for treating Restless Legs Syndrome (RLS).

It is particularly preferred to use an NK₁-receptor antagonist selected from among BIIF 1149, CGP 60829, MK-869, CJ-11974 and GR 205171, for preparing a pharmaceutical composition for treating Restless Legs Syndrome (RLS), while the use of BIIF 1149 may be regarded as particularly preferred.

In every case, a pharmacologically acceptable salt or an ester or a prodrug form, e.g. an ester, of the above-mentioned compounds may be used as the active substance.

If desired, the use of NK₁-receptor antagonists according to the invention in the treatment of Restless Legs Syndrome may be combined with the administration of other active substances in order to achieve a synergistic therapeutic effect. According to another aspect, the present invention therefore relates to the use of a combination of active substances for the preparation of a pharmaceutical composition or pharmaceutical kit for the treatment of Restless Legs Syndrome, characterised in that at least one of the active substances contained in the pharmaceutical composition or pharmaceutical kit is an NK₁-receptor antagonist and in that moreover it contains at least one active substance selected from among the α2-agonists, opioids, benzodiazepines, anti-Parkinson's agents, preferably dopamine agonists, levodopa (L-dopa) plus decarboxylase inhibitors and NK3-receptor antagonists.

Within the scope of the combinations of active substances mentioned above, imidazole receptor agonists may preferably be used as the α2-agonists. The following are particularly preferred: agmatine, apraclonidine, azepexole, clonidine, dexmedetomidine, guanfacinel, guanabenz, lofexidine, medetomidine, naphazoline, oxymetazoline, para-amino-clonidine, rilmenidine, romifidine, talipexole, teryzoline, tiamenidine, tinabinol, tizanidine, tolonidine, xylometazoline, xylazine, AGN-190837, AGN-192836, BAM-1125, CP-18534-1, DJ-741, ICI-106270, IDPH-791, MPV-295, MPV-2426, RWJ-52807, S-18616, ST-91, U-47476A, UK-1403, UK-14304, 6-(5-methyl-quinoxalinyl)-imino-imidazolidine. Of these, the ones of particular interest for preparing the combination of active substances according to the invention are agmatine, apraclonidine, azepexole, clonidine, dexmedetomidine, guanfacinel, guanabenz, lofexidine, naphazoline, oxymetazoline, para-amino-clonidine, rilmenidine, romifidine, talipexole, teryzoline, tiamenidine, tinabinol, tizanidine, tolonidine, xylometazoline, xylazine and S-18616, clonidine being particularly preferred. In every case, a pharmacologically acceptable salt or an ester or a prodrug form, e.g., an ester, of the above-mentioned compounds may also be used as the active substance.

Of the active substance components levodopa (L-dopa) plus decarboxylase inhibitor which may also be used to prepare the combinations of active substances according to the invention containing at least one NK1-receptor antagonist, the combinations of L-dopa with benserazide and L-dopa with carbidopa are particularly preferred.

Of the dopamine agonists, bromocryptine, cabergoline, α-dihydroergocryptine, lisuride, pergolide, pramipexole (HCl), talipexole, ropinirol, S(-)-2-(N-propyl-N-2-thienylethylamino)-5-hydroxy-tetraline (e.g. as N-0923) or (R)-5,6-dihydro-5-(methylamino)-4H-imidazo(4,5-ij)-quinolin-2(1H)-one R-6 (PNU 95666) or a pharmacologically acceptable salt thereof are preferred.

Of the opioids, buprenorphine, codeine, dextropropoxyphen, dihydrocodeine, fentanyl, hydromorphone, levomethadone, morphine, oxycodon, pethidine, tilidine, tramadol or the pharmacologically acceptable salts thereof are preferred. Particularly preferred are codeine, dihydrocodeine, tramadol, sufentanil and morphine.

Of the benzodiazepines, clonazepam and brotizolam are preferred.

Within the scope of the above-mentioned active substance combinations, SR-142801 (Osanetant) is preferably used as the NK₃-receptor antagonist.

The combination of the NK₁-receptor antagonists which may be used according to the invention with clonidine or one of the pharmacologically acceptable salts thereof, or with a dopamine antagonist, preferably with pramipexole, or a pharmacologically acceptable salt thereof, is preferred.

The NK₁-receptor antagonists which may be used according to the invention may be formulated according to the current pharmaceutical methods known from the prior art so that they can be administered by oral, spinal, epidural, anal or intravenous route or by inhalation, subcutaneously or transdermally. Oral and transdermal preparations are preferred. The same applies to the other active substances mentioned hereinbefore which may optionally be contained in the preparation to achieve a synergistic effect. The daily dose to be administered will naturally depend on the extent and seriousness of the RLS symptoms. According to the invention, the broad dosage range which may be used is about 20-500 mg of the NK₁-receptor antagonist per day.

The preparation may be given orally in the form of a tablet, powder, powder in a capsule (e.g. a hard gelatine capsule), as a solution or suspension. For spinal, intravenous and subcutaneous applications, the combination of active substances according to the invention is given as a solution. The preparation may be administered anally in suppositories. For inhalation, the combination of active substances may be given as a powder, as an aqueous or aqueous-ethanolic solution or using a propellant gas formulation. For transdermal administration the active substance may be applied to the skin as an ointment or cream, but is preferably applied by means of a plaster.

In the case of plasters, the active substance or combination of active substances is either released directly onto the outer layer of the skin or is released directly into the underlying layers of the skin using a transdermal plaster, in the form of a solution or a gel, e.g. embedded in a polymer matrix, through micro-needles or micro-cutters which penetrate the horny layer of the skin. A transdermal plaster with micro-pins or micro-cutters of this kind is disclosed for example in patent application WO 97/03718. Patent application WO 91/07998 describes a process by means of which active substances can be applied more satisfactorily transdermally by adjusting the skin to a specific pH. U.S. Pat. No. 5,112,842, or the corresponding European Patent EP 0428038, discloses a transdermal plaster for administering pramipexole. Reference is hereby made expressly to the contents of all three patents, to show how the combination of active substances according to the invention can be applied using a transdermal plaster.

Both types of plaster described above (with and without microcutters or micropins) release the active substance continuously onto or into the skin, so as to avoid concentration peaks and the possible side effects associated with them. The active substance or combination of active substances can be released passively or actively. Active transfer can be by purely mechanical means, electrically, osmotically or by iontophoresis. If desired, the release may be controlled electronically, optionally with monitoring of the blood plasma level by sensors or microsensors which are integrated in the plaster or communicate therewith, as a result of which the blood plasma level can be adjusted deliberately to suit individual requirements and consequently a steady release is not absolutely essential.

In those cases where NK₁-receptor antagonists are to be used within the framework of a combination therapy with the other active substances mentioned above, the combinations of active substances may be formulated separately (e.g. in a capsule or as a tablet), in a single formulation but separate from one another (e.g. in a capsule with two or more chambers) or mixed together in a single formulation (e.g. in the form of a tablet or in a capsule with only one chamber). When the active substances are formulated separately independently of one another, it is not essential for the two substances to be administered by the same route of administration; rather, combinations of formulations may be used wherein the two active substances are administered by separate routes. However, those formulations wherein the two active substances are administered by the same route are preferred. The two active substances are advantageously administered together in one preparation.

In the case of the transdermal plasters, the active substances may be administered, for example, either in separate plasters, in a joint plaster in which the active substances are stored separately within the plaster, or they may be mixed together in one plaster. The same is also true of the other administration forms described above.

The active substance formulation according to the invention is prepared by the methods known from the prior art, depending on the method of administration, and may accordingly contain the formulation constituents known in the art.

They may also contain other pharmacologically active substances or cosmetic additives.

In every case, the NK₁-receptor antagonists as well as any other active substances additionally provided in order to achieve a synergistic effect may be used both as neutral compounds or in the form of a pharmacologically acceptable salt. The NK₁-receptor antagonists as well as any other active substances additionally provided in order to achieve a synergistic effect may be used both as neutral compounds and as identical or different salts or as a combination of a salt of an active substance and other, neutral, active substances. The different variants are influenced by the method of administration. Where the NK₁-receptor antagonists as well as any other active substances additionally provided in order to achieve a synergistic effect are provided in a joint formulation, this is preferably the neutral compound or the same salt (e.g. the hydrochloride). The same is also preferably true when the NK₁-receptor antagonists as well as any other active substances additionally provided in order to achieve a synergistic effect are taken orally as tablets or capsules.

Independently of the method of administration, the NK₁-receptor antagonists as well as any other active substances additionally provided in order to achieve a synergistic effect are preferably administered within a time frame of preferably 24 hours, most preferably within 12 hours and particularly within 1 hour of each other. Most prefereably, they should be administered simultaneously, within not more than 15 minutes of each other.

The present invention is not to be limited in scope by the exemplified embodiments which are intended as illustrations of single aspects of the invention. Indeed various modifications of the invention in addition to those shown and described herein will become apparent to those skilled in the art from the foregoing description. Such modifications are intended to fall within the scope of the appended claims.

All publications and patent applications cited herein are incorporated by reference in their entireties.

What is claimed is:

1. A method for treating Restless Legs Syndrome (RLS) comprising administering a neurokinin 1 (NK₁)-receptor antagonist.

2. A method for treating Restless Legs Syndrome (RLS) comprising administering a neurokinin 1 (NK₁)-receptor antagonist, wherein the NK₁-receptor antagonist is selected from the group consisting of BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-I, CJ-11974, TAK-637, and GR 205171.

3. The method according to claim 1 or 2, wherein the NK₁-receptor antagonist is selected from the group consisting of BIIF 1149, CGP 60829, MK-869, CJ-11974, and GR 205171.

4. The method according to claim 3, wherein the NK₁-receptor antagonist is in the form of a pharmacologically acceptable salt, an ester or a prodrug.

5. The method according to claim 1 or 2, further comprising administering an active substance, wherein the combination of the NK₁-receptor antagonist and the active substance produces a synergistic therapeutic effect.

6. The method according to claim 1 or 2, further comprising administering an active substance, wherein the active substance is selected from the group consisting of α2-agonists, opioids, benzodiazepines, anti-Parkinson's agents, levodopa (L-dopa) in combination with a decarboxylase inhibitor, and NK₃-receptor antagonists; and wherein the combination of the NK₁-receptor antagonist and the active substance produces a synergistic therapeutic effect.

7. A pharmaceutical composition comprising the neurokinin 1 (NK₁)-receptor antagonist of claim 1 or 2; and a pharmaceutically acceptable carrier.

8. The pharmaceutical composition of claim 7, further comprising an active substance, wherein the active substance is selected from the group consisting of α2-agonists, opioids, benzodiazepines, anti-Parkinson's agents, levodopa (L-dopa) in combination with a decarboxylase inhibitor, and NK₃-receptor antagonists.
